(19) United States
(12) Patent Application Publication
(10) Pub. No.: US 2017/0281933 A1
(43) Pub. Date: Oct. 5, 2017

(54) SPINAL CORD STIMULATION LEADS WITH CENTRALLY-CONCENTRATED CONTACTS, AND ASSOCIATED SYSTEMS AND METHODS

(71) Applicant: Nevro Corp., Redwood City, CA (US)

(72) Inventor: Marc Russo, The Junction (AU)

(21) Appl. No.: 15/090,580

(22) Filed: Apr. 4, 2016

(51) Int. Cl. A61N 1/06; A61N 1/36; A61N 1/05

(52) U.S. Cl. CPC A61N 1/06; A61N 1/0551; A61N 1/36071; A61N 1/36171

(57) ABSTRACT

Spinal cord stimulation leads with centrally-concentrated contacts, and associated systems and methods. A representative lead system includes a proximal portion and a distal portion, a plurality of signal delivery contacts carried by the distal portion, with multiple distal-most signal delivery contacts spaced apart by a first distance, multiple proximal-most signal delivery contacts spaced apart by a second distance, and multiple intermediate signal delivery contacts spaced apart by a third distance less than the first and second distances. A plurality of connection contacts is carried by the proximal portion, and individual conductors are connected between individual connection contacts and corresponding individual signal delivery contacts.

### TECHNICAL FIELD

The present technology is directed generally to spinal cord stimulation leads with centrally-concentrated contacts, and associated systems and methods. Particular embodiments include leads having contacts specifically positioned to direct electrical signals to a narrow segment of the spinal cord, for example, at approximately the T9-T10 disc location.

### BACKGROUND

Neurological stimulators have been developed to treat pain, movement disorders, functional disorders, spasticity, cancer, cardiac disorders, and various other medical conditions. Implantable neurological stimulation systems generally have an implantable signal generator and one or more leads that deliver electrical pulses to neurological tissue or muscle tissue. For example, several neurological stimulation systems for spinal cord stimulation (SCS) have cylindrical leads that include a lead body with a circular cross-sectional shape and one or more conductive rings (i.e., contacts) spaced apart from each other at the distal end of the lead body. The conductive rings operate as individual electrodes and, in many cases, the SCS leads are implanted percutaneously through a needle inserted into the epidural space, with or without the assistance of a stylet.

Once implanted, the signal generator applies electrical pulses to the electrodes, which in turn modify the function of the patient's nervous system, such as by altering the patient's responsiveness to sensory stimuli and/or altering the patient's motor-circuit output. In SCS therapy for the treatment of pain, the signal generator applies electrical pulses to the spinal cord via the electrodes. In conventional SCS therapy, electrical pulses are used to generate sensations (known as paresthesia) that mask or otherwise alter the patient's sensation of pain. For example, in many cases, patients report paresthesia as a tingling sensation that is perceived as less uncomfortable than the underlying pain sensation.

In contrast to traditional or conventional (i.e., paresthesia-based) SCS, forms of paresthesia-free SCS have been developed that use therapy signal parameters that treat the patient's sensation of pain without generating paresthesia or otherwise using paresthesia to mask the patient's sensation of pain. One of several advantages of paresthesia-free SCS therapy systems is that they eliminate the need for uncomfortable paresthesias, which many patients find objectionable. Nevertheless, there remains a need for delivering spinal cord therapy signals in as efficient a manner as possible, for example, to reduce the amount of time required to determine an optimal location for the patient, and/or to reduce the amount of power required to produce the therapy signal.

### BRIEF DESCRIPTION OF THE DRAWINGS

FIG. 1A is a partially schematic illustration of an implantable spinal cord modulation system positioned at the spine to deliver therapeutic signals in accordance with several embodiments in the present technology.

FIG. 1B is a partially schematic, cross-sectional illustration of a patient's spinal cord region, illustrating representative locations for implanted lead bodies in accordance with embodiments of the present technology.

FIG. 2 is a partially schematic illustration of a patient's spinal column, illustrating vertebrae and intervertebral discs.

FIG. 3 is a partially schematic, cross-sectional illustration of a representative intervertebral disc and associated neural structures.

FIG. 4 is a partially schematic illustration of a portion of the patient's spinal column, with a lead positioned in accordance with an embodiment of the present technology.

FIG. 5 is a partially schematic, isometric illustration of a lead system including a lead having contacts spaced in accordance with an embodiment of the present technology.

FIG. 6 is a partially schematic illustration of a lead system that includes a lead and a lead extension having bifurcated connection portions in accordance with an embodiment of the present technology.

FIG. 7 is a partially schematic illustration of a lead system that includes a lead having bifurcated connection portions in accordance with an embodiment of the present technology.

### DETAILED DESCRIPTION

The present technology is directed generally to the spinal cord stimulation leads with centrally-concentrated contacts, and associated systems and methods. The centrally-concentrated contacts can increase the likelihood for targeting specific locations of the spinal cord expected to produce particularly efficacious therapy for patients receiving paresthesia-free spinal cord stimulation (SCS) For example, in one embodiment, the centrally-concentrated contacts can be positioned epidurally at a location aligned (along a rostral-caudal axis) with the T9-T10 intervertebral disc (i.e., the disc located between the T9 and T10 vertebrae). This location may be particularly effective for addressing a patient's low back pain because neural pathways from the lower back enter the dorsal horn of the spinal cord at this location. Accordingly, leads with electrical contacts concentrated in a region of the lead that may be readily positioned at the T9-T10 disc can be particularly effective for addressing a patient's low back pain. As will be described in further detail below, it is expected that discogenic pain signals associated with low back pain and generated much lower in the spinal column (e.g., at the lumbar vertebral levels), enter the spinal cord at about the same level as the T9-T10 disc. Accordingly, sizing, positioning, and/or spacing the contacts on the lead (or other signal delivery device) in such a way that more contacts are positioned along the spinal cord at about the location of the T9-T10 disc, can increase the likelihood for successfully targeting the patient's pain. This approach can be particularly useful in cases for which the lead (or other device) migrates over time, for example, between the time the patient first tries out the device, and the time the patient receives a long-term or permanent implant.

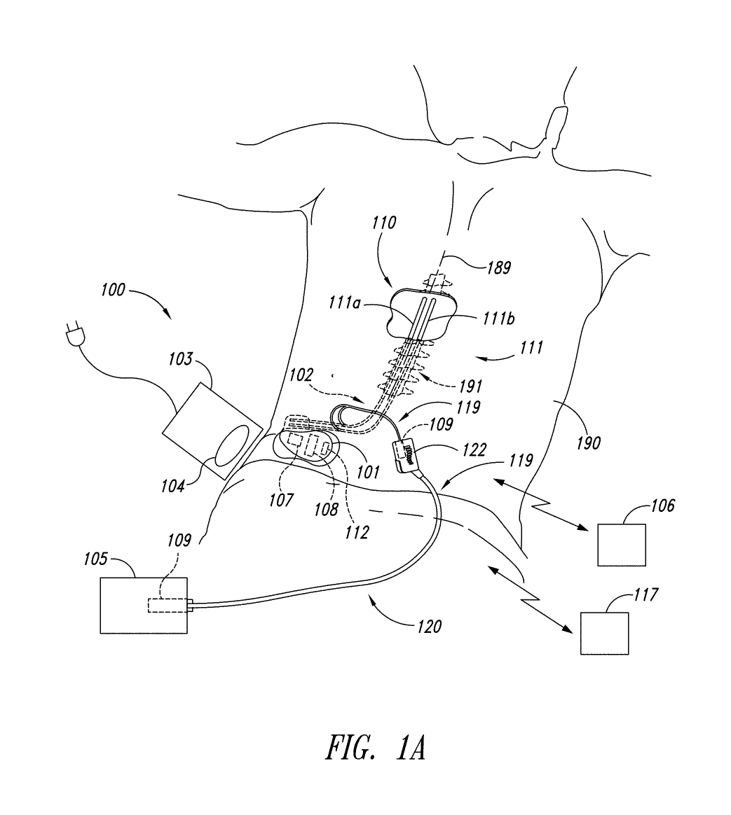

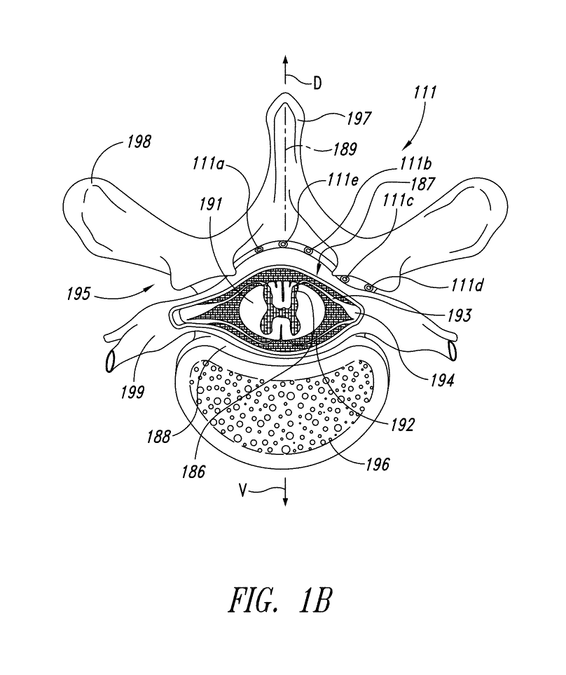

General aspects of the environments in which the disclosed technology operates are described below under Heading 1.0 (“Overview”) with reference to FIGS. 1A and 1B. Particular embodiments of the technology along with more specific details of the spinal column structure are described further under Heading 2.0 (“Representative Embodiments”) with reference to FIGS. 2-7. Additional embodiments are described under Heading 3.0 (“Additional Embodiments”). While the present technology is described in the environment of SCS, one with skill in the art would recognize that one or more aspects of the present technology are applicable to other, non-SCS implantable devices; e.g., more generally, implantable neurostimulators for treatment of one or more patient indications.

### 1.0 Overview

One example of a paresthesia-free SCS therapy system is a “high frequency” SCS system. High frequency SCS systems can inhibit, reduce, and/or eliminate pain via waveforms with high frequency elements or components (e.g., portions having high fundamental frequencies), generally with reduced or eliminated side effects. Such side effects can include unwanted paresthesia, unwanted motor stimulation or blocking, unwanted pain or discomfort, and/or interference with sensory functions other than the targeted pain. In a representative embodiment, a patient may receive high frequency therapeutic signals with at least a portion of the therapy signal at a frequency of from about 1.5 kHz to about 100 kHz, or from about 1.5 kHz to about 50 kHz, or from about 3 kHz to about 20 kHz, or from about 5 kHz to about 15 kHz, or from about 1.5 kHz to about 10 kHz, or at frequencies of about 8 kHz, 9 kHz, or 10 kHz. These frequencies are significantly higher than the frequencies associated with conventional “low frequency” SCS, which are generally below 1,200 Hz, and more commonly below 100 Hz. Accordingly, modulation at these and other representative frequencies (e.g., from about 1.5 kHz to about 100 kHz) is occasionally referred to herein as “high frequency stimulation,” “high frequency SCS,” and/or “high frequency modulation.” Further examples of paresthesia-free SCS systems are described in U.S. Patent Publication Nos. 2009/0204173 and 2010/0274314, the respective disclosures of which are herein incorporated by reference in their entireties. In further embodiments, the approach discussed herein can be applied to other methods for applying therapy signals, e.g., in cases for which the likely location of a particularly efficacious (e.g., optimal) stimulation site follows a normal or approximately normal distribution.

FIG. 1A schematically illustrates a representative patient therapy system 100 (e.g., a spinal cord stimulator) for providing relief from chronic pain and/or other conditions, arranged relative to the general anatomy of a patient's spinal column 191. The system 100 can include a signal generator 101 (e.g., an implanted or implantable pulse generator or IPG), which may be implanted subcutaneously within a patient 190 and coupled to one or more signal delivery elements or devices 110. The signal delivery elements or devices 110 may be implanted within the patient 190, typically at or near the patient's spinal cord midline 189. The signal delivery elements 110 carry features for delivering therapy to the patient 190 after implantation. The signal generator 101 can be connected directly to the signal delivery devices 110, or it can be coupled to the signal delivery devices 110 via a signal link or lead extension 102. In a further representative embodiment, the signal delivery devices 110 can include one or more elongated lead(s), which can in turn include corresponding lead bodies. The lead or other signal delivery device 110, alone or in combination with other elements (e.g., the extension 102), are sometimes referred to as a lead system 119. As used herein, the terms signal delivery device, lead, and/or lead body include any of a number of suitable substrates and/or support members that carry electrodes/devices for providing therapy signals to the patient 190. For example, the lead or leads can include one or more electrodes or electrical contacts that direct electrical signals into the patient's tissue, e.g., to provide for therapeutic relief. In other embodiments, the signal delivery elements 110 can include structures other than a lead body (e.g., a paddle) that also direct electrical signals and/or other types of signals to the patient 190. In any of these embodiments, the leads can form a portion of a lead system, which may include other elements e.g., signal links or lead extensions, which are discussed further below.

In a representative embodiment, one signal delivery device may be implanted on one side of the spinal cord midline 189, and a second signal delivery device may be implanted on the other side of the spinal cord midline 189. For example, first and second leads 111a, 111b shown in FIG. 1A may be positioned just off the spinal cord midline 189 (e.g., about one millimeter offset) in opposing lateral directions so that the two leads 111a, 111b are spaced apart from each other by about two millimeters. In particular embodiments, the leads 111 may be implanted at a vertebral level ranging from, for example, about T8 to about T12. In other embodiments, one or more signal delivery devices can be implanted at other vertebral levels, e.g., as disclosed in U.S. Patent Application Publication No. 2013/0066411, which is incorporated herein by reference in its entirety.

The signal generator 101 can transmit signals (e.g., electrical signals) to the signal delivery elements 110 that up-regulate (e.g., excite) and/or down-regulate (e.g., block or suppress) target nerves. As used herein, and unless otherwise noted, the terms “modulate,” “modulation,” “stimulate,” and “stimulation” refer generally to signals that have either type of the foregoing effects on the target nerves. The signal generator 101 can include a machine-readable (e.g., computer-readable or controller-readable) medium containing instructions for generating and transmitting suitable therapy signals. The signal generator 101 and/or other elements of the system 100 can include one or more processor(s) 107, memory unit(s) 108, and/or input/output device(s) 112. Accordingly, the process of providing modulation signals, providing guidance information for positioning the signal delivery devices 110, establishing battery charging and/or discharging parameters, and/or executing other associated functions can be performed by computer-executable instructions contained by, on or in computer-readable media located at the pulse generator 101 and/or other system components. Further, the pulse generator 101 and/or other system components may include dedicated hardware, firmware, and/or software for executing computer-executable instructions that, when executed, perform any one or more of the methods, processes, and/or sub-processes described herein. The dedicated hardware, firmware, and/or software also serve as “means for” performing the methods, processes, and/or sub-processes described herein. The signal generator 101 can also include multiple portions, elements, and/or subsystems (e.g., for directing signals in accordance with multiple signal delivery parameters), carried in a single housing, as shown in FIG. 1A, or in multiple housings.

The signal generator 101 can also receive and respond to an input signal received from one or more sources. The input signals can direct or influence the manner in which the therapy, charging, and/or process instructions are selected, executed, updated, and/or otherwise performed. The input signals can be received from one or more sensors (e.g., an input device 112 shown schematically in FIG. 1A for purposes of illustration) that are carried by the signal generator 101 and/or distributed outside the signal generator 101 (e.g., at other patient locations) while still communicating with the signal generator 101. The sensors and/or other input devices 112 can provide inputs that depend on or reflect patient state (e.g., patient position, patient posture, and/or patient activity level), and/or inputs that are patient-independent (e.g., time). Still further details are included in U.S. Pat. No. 8,355,797, incorporated herein by reference in its entirety.

In some embodiments, the signal generator 101 and/or signal delivery devices 110 can obtain power to generate the therapy signals from an external power source 103. In one embodiment, for example, the external power source 103 can by-pass an implanted signal generator and generate a therapy signal directly at the signal delivery devices 110 (or via signal relay components). The external power source 103 can transmit power to the implanted signal generator 101 and/or directly to the signal delivery devices 110 using electromagnetic induction (e.g., RF signals). For example, the external power source 103 can include an external coil 104 that communicates with a corresponding internal coil (not shown) within the implantable signal generator 101, signal delivery devices 110, and/or a power relay component (not shown). The external power source 103 can be portable for ease of use.

In another embodiment, the signal generator 101 can obtain the power to generate therapy signals from an internal power source, in addition to or in lieu of the external power source 103. For example, the implanted signal generator 101 can include a non-rechargeable battery or a rechargeable battery to provide such power. When the internal power source includes a rechargeable battery, the external power source 103 can be used to recharge the battery. The external power source 103 can in turn be recharged from a suitable power source (e.g., conventional wall power).

During at least some procedures, an external stimulator or trial modulator 105 can be coupled to the signal delivery elements 110 during an initial procedure, prior to implanting the signal generator 101. For example, a practitioner (e.g., a physician and/or a company representative) can use the trial modulator 105 to vary the modulation parameters provided to the signal delivery elements 110 in real time, and select optimal or particularly efficacious parameters. These parameters can include the location from which the electrical signals are emitted, as well as the characteristics of the electrical signals provided to the signal delivery devices 110. In some embodiments, input is collected via the external stimulator or trial modulator and can be used by the clinician to help determine what parameters to vary. In a typical process, the practitioner uses a cable assembly 120 to temporarily connect the trial modulator 105 to the signal delivery device 110. The practitioner can test the efficacy of the signal delivery devices 110 in an initial position. The practitioner can then disconnect the cable assembly 120 (e.g., at a connector 122), reposition the signal delivery devices 110, and reapply the electrical signals. This process can be performed iteratively until the practitioner obtains the desired position for the signal delivery devices 110. Optionally, the practitioner may move the partially implanted signal delivery devices 110 without disconnecting the cable assembly 120. Furthermore, in some embodiments, the iterative process of repositioning the signal delivery devices 110 and/or varying the therapy parameters may not be performed.

The signal generator 101, the lead extension 102, the trial modulator 105 and/or the connector 122 can each include a receiving element 109. Accordingly, the receiving elements 109 can be patient implantable elements, or the receiving elements 109 can be integral with an external patient treatment element, device or component (e.g., the trial modulator 105 and/or the connector 122). The receiving elements 109 can be configured to facilitate a simple coupling and decoupling procedure between the signal delivery devices 110, the lead extension 102, the pulse generator 101, the trial modulator 105 and/or the connector 122. The receiving elements 109 can be at least generally similar in structure and function to those described in U.S. Patent Application Publication No. 2011/0071593, incorporated by reference herein in its entirety.

After the signal delivery elements 110 are implanted, the patient 190 can receive therapy via signals generated by the trial modulator 105, generally for a limited period of time. During this time, the patient wears the cable assembly 120 and the trial modulator 105 outside the body. Assuming the trial therapy is effective or shows the promise of being effective, the practitioner then replaces the trial modulator 105 with the implanted signal generator 101, and programs the signal generator 101 with therapy programs selected based on the experience gained during the trial period. Optionally, the practitioner can also replace the signal delivery elements 110. Once the implantable signal generator 101 has been positioned within the patient 190, the therapy programs provided by the signal generator 101 can still be updated remotely via a wireless physician's programmer (e.g., a physician's laptop, a physician's remote or remote device, etc.) 117 and/or a wireless patient programmer 106 (e.g., a patient's laptop, patient's remote or remote device, etc.). Generally, the patient 190 has control over fewer parameters than does the practitioner. For example, the capability of the patient programmer 106 may be limited to starting and/or stopping the signal generator 101, and/or adjusting the signal amplitude. The patient programmer 106 may be configured to accept pain relief input as well as other variables, such as medication use.

FIG. 1B is a cross-sectional illustration of the spinal cord 191 and an adjacent vertebra 195 (based generally on information from Crossman and Neary, “Neuroanatomy,” 1995 (published by Churchill Livingstone)), along with multiple leads 111 (shown as leads 111a-111e) implanted at representative locations. For purposes of illustration, multiple leads 111 are shown in FIG. 1B implanted in a single patient. In actual use, any given patient will likely receive fewer than all the leads 111 shown in FIG. 1B.

The spinal cord 191 is situated within a vertebral foramen 188, between a ventrally located ventral body 196 and a dorsally located transverse process 198 and spinous process 197. Arrows V and D identify the ventral and dorsal directions, respectively. The spinal cord 191 itself is located within the dura mater 199, which also surrounds portions of the nerves exiting the spinal cord 191, including the ventral roots 192, dorsal roots 193 and dorsal root ganglia 194. The dorsal roots 193 enter the spinal cord 191 at the dorsal root entry zone 187, and communicate with dorsal horn neurons located at the dorsal horn 186. In one embodiment, the first and second leads 111a, 111b are positioned just off the spinal cord midline 189 (e.g., about one millimeter. offset) in opposing lateral directions so that the two leads 111a, 111b are spaced apart from each other by about two millimeters, as discussed above. In other embodiments, a lead or pairs of leads can be positioned at other locations, e.g., toward the outer edge of the dorsal root entry zone 187 as shown by a third lead 111c, or at the dorsal root ganglia 194, as shown by a fourth lead 111d, or approximately at the spinal cord midline 189, as shown by a fifth lead 111e.

### 2.0 Representative Embodiments

FIGS. 2-4 illustrate further details of the patient's spinal column 191 and associated structures, which form the environment in which leads and lead systems in accordance with the present technology operate. FIG. 2 illustrates the patient's spinal column 191, including cervical vertebrae C1-C7, thoracic vertebrae T1-T12, lumbar vertebrae L1-L5, and the sacrum (S1-S5) and coccyx. Corresponding discs 284 are located between neighboring vertebrae to provide cushioning, among other functions. In many patients suffering from low back pain, the pain originates at the L4-L5 disc 284a.

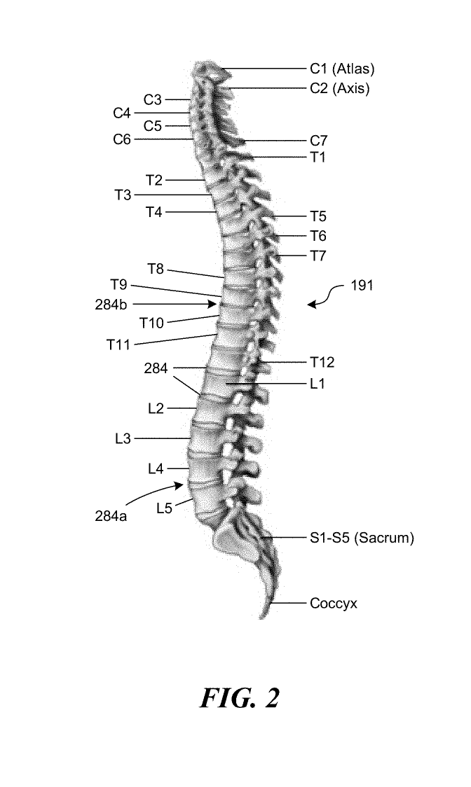

FIG. 3 is a partially schematic cross-sectional illustration of a representative disc 284 (e.g., the L4-L5 disc 284a described above with reference to FIG. 2). The disc 284 includes a nucleus pulposus 383 surrounded by an annulus fibrosus 382. A posterior disc plexus 381 receives neural impulses from a multitude of small nerves that innervate the disc 284 and are referred to herein collectively as disc innervation nerves 380. The posterior disc plexus 381 transmits afferent signals from the disc innervation nerves 380 via the grey ramus communicans 379. The grey ramus communicans 379 transmits the afferent signals to the sympathetic trunk 378, which extends upwardly and downwardly along the spinal column 191, e.g., into and out of the plane of FIG. 3.

Following an injury to the disc 284, discogenic pain can be caused when the nucleus pulposus 383 leaks outwardly as neovascularization causes further innervation to extend inwardly into the disc 284. The patient's pain can result because interleukin 6 in the nucleus pulposus 383 can cause inflammation of the nearby neurons, particularly the disc innervation nerves 380. Afferent pain signals caused by the inflammation travel to the sympathetic trunk 378 via the posterior disc plexus 381 and the grey ramus communicans 379, as described above. Signals generated between the L5 and L2 vertebrae all enter the spinal column 191 at the T11 vertebral level (FIG. 2). These neurons then synapse with the wide dynamic range neurons located at the dorsal horn 186 (FIG. 1B), one and a half vertebral segments above T11. This region of the spinal cord is aligned generally (in the rostral/caudal direction) with the T9-T10 disc 284b (FIG. 2). Accordingly, electrical stimulation provided to the spinal cord at a location aligned or approximately aligned with the T9-T10 disc 284b can address discogenic pain that originates at the L2-L5 vertebral levels.

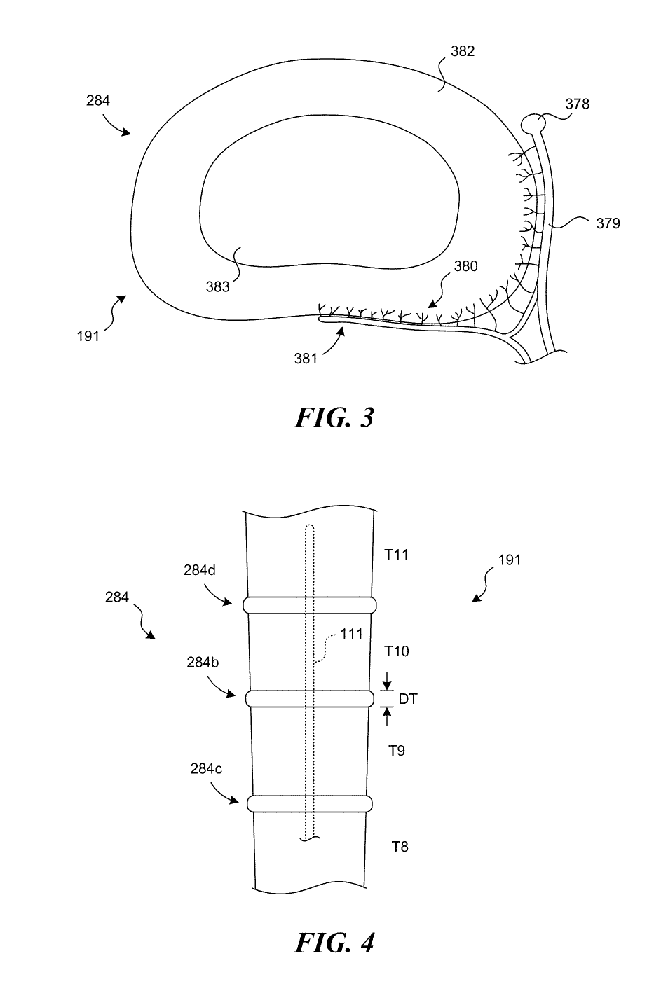

FIG. 4 is a partially schematic illustration of a portion of the spinal column 285 illustrating the T8-T11 vertebrae, and the associated T8-T9 disc 284c, the T9-T10 disc 284b, and the T10-T11 disc 284d. The individual discs 284 can have a disc thickness DT in a range from about three millimeters to about fifteen millimeters. The neural pathways from the lumbar region may enter the dorsal column at locations not precisely aligned with the T9-T10 disc 284b, and may instead enter at a location aligned with the lower portion of the T10 vertebrae or the upper portion of the T9 vertebrae. Accordingly, the lead 111 can include signal delivery contacts that are concentrated in the region of the T9-T10 disc 284b, and regions just above and below the T9-T10 disc 284b.

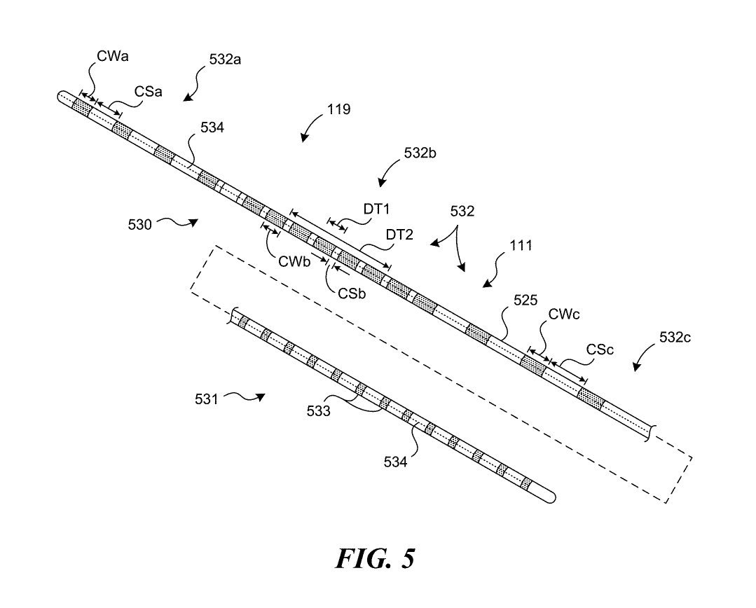

FIG. 5 is a partially schematic, enlarged view of the lead 111 shown in FIG. 4. For purposes of illustration, FIG. 5 also shows a representative minimum disc thickness DT1 (e.g., three millimeters) and a representative maximum disc thickness DT2 (e.g., about fifteen millimeters). The lead 111 includes a distal portion 530, which is positioned adjacent the target area for stimulation, and a proximal portion 531, which is connected directly or indirectly to the pulse generator 101 (described above with reference to FIG. 1A). The lead 111 includes a lead body 525 carrying multiple signal delivery contacts 532 which are shaded for purposes of illustration. The signal delivery contacts 532 can be sized and/or spaced differently depending on where within the distal portion 530 they are located. For example, a group of distal-most signal delivery contacts 532a can each have a first contact width CWa and can be spaced apart from each other by a first contact spacing CSa. Intermediate signal delivery contacts 532b can have a second contact width CWb and a second contact spacing CSb, and a group of proximal-most signal delivery contacts 532c can have a corresponding third contact width CWc and a third contact spacing CSc. Representative specific examples are provided below.

In several embodiments, and as is shown in FIG. 5, the intermediate signal delivery contacts 532b can have a second contact spacing CSb, which is less than the first contact spacing CSa of the distal-most signal delivery contacts 532a, and less than the third contact spacing CSc of the proximal-most signal delivery contacts 532c. In particular embodiments, each of the signal delivery contacts 532 can have the same width while the contact spacings can vary from one group to another. In particular, the intermediate signal delivery contacts 532b can have a second contact spacing CSb of three millimeters or less in one embodiment, less than two millimeters in another embodiment, and one millimeter in still another embodiment. It is expected that contact spacings of less than one millimeter may cause adjacent signal delivery contacts to electrically “short” with each other due to the low impedance between signal delivery contacts with such a close spacing. In some cases, even signal delivery contacts spaced apart by one millimeter or more may electrically short with each other. In such instances, the practitioner can skip a signal delivery contact when establishing bipolar pairs of signal delivery contacts for therapy. Accordingly, the second contact spacing CSb can be less than the spacing between other signal delivery contacts on the signal delivery device, and in particular embodiments, less than 2/3 of the spacing between other such contacts, or less than 1/2 the spacing between other such contacts, or less than 1/3 the spacing between other such contacts.

The three millimeters signal delivery contact width can advantageously provide consistency and commonality with conventional contacts. However, in other embodiments, the contact widths can be greater or less than three millimeters, and can vary from one group to another. Importantly, the intermediate signal delivery contacts 532b are spaced more closely together so as to provide the practitioner with more options for delivering signals to the intervertebral disc region, represented by disc thickness dimensions DT1 and DT2.

The proximal portion 531 of the lead 111 includes connection contacts 533 (also shaded for purposes of illustration) that are used to connect the lead 111 directly or indirectly to the signal generator 101 (FIG. 1A). In a particular embodiment, multiple conductors 534 each connect an individual signal delivery contact 532 with a corresponding connector contact 533. For purposes of illustration, the multiple conductors 534 are shown in FIG. 5 as a single, axially extending dotted line. In a particular embodiment shown in FIG. 5, the lead 111 includes sixteen signal delivery contacts 532, sixteen corresponding connection contacts 533 and sixteen corresponding conductors 534.

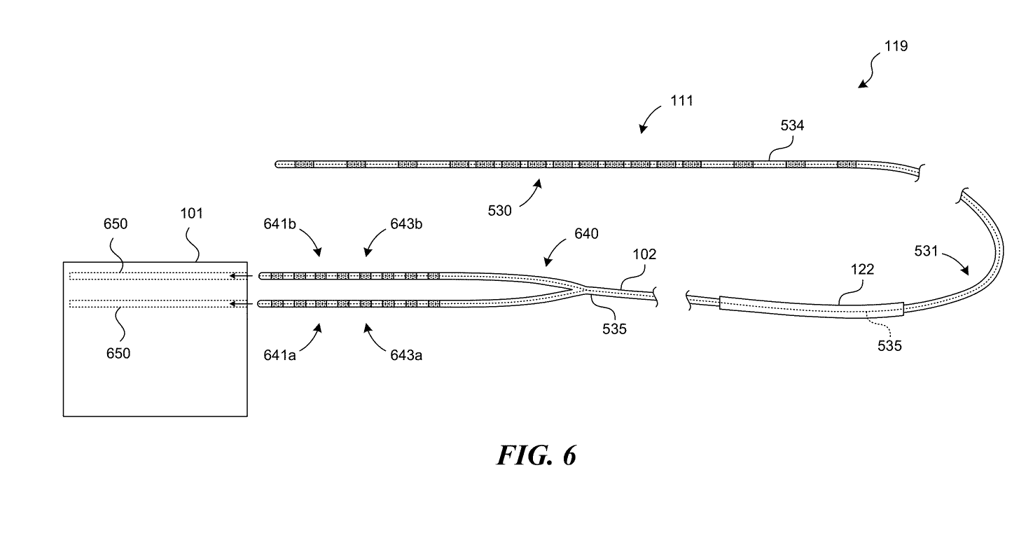

FIG. 6 is a partially schematic illustration of an embodiment of the lead system 119 in which the lead 111 is coupled to the corresponding signal generator 101 with a lead extension 102. The proximal portion 531 of the lead 111 is inserted into a connector 122 carried by the lead extension 102. The lead extension 102 can include a bifurcation 640 at which the lead extension 102 separates into a first connection portion 641a and a second connection portion 641b. Each portion can carry corresponding extension contacts 643, illustrated as first extension contacts 643a and second extension contacts 643b. This arrangement can be used when the signal generator 101 has two connection ports 650, each of which has up to eight terminals to accommodate up to eight extension contacts 643. Accordingly, when the lead 111 includes sixteen signal delivery contacts 532 as described above, and the lead extension 102 includes a corresponding sixteen extension contacts 643, all sixteen extension contacts can be accommodated by the two connection ports of the signal generator 101.

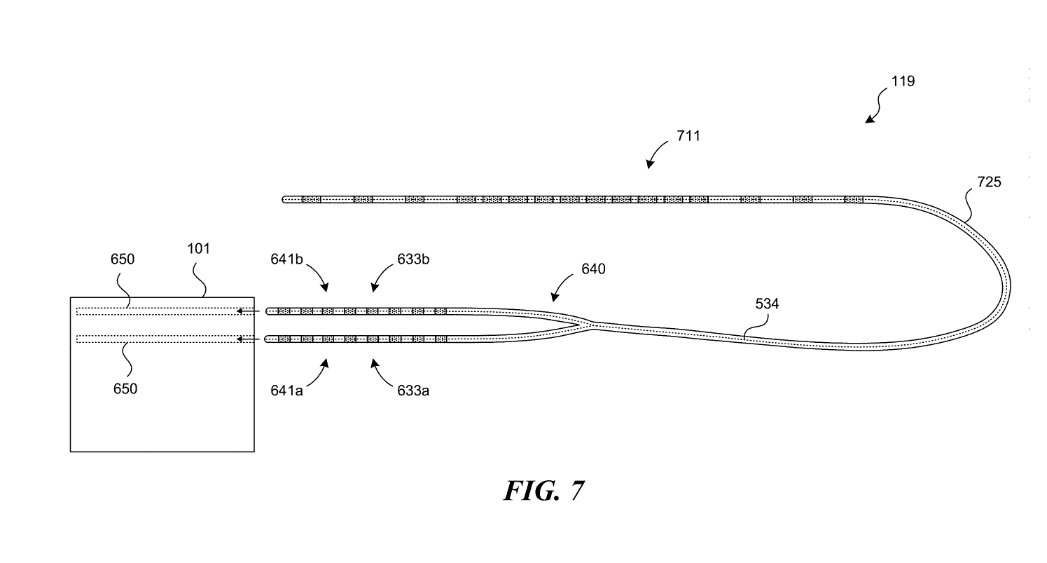

In another embodiment, illustrated in FIG. 7, the lead system 119 can include a lead 711 that connects directly to the signal generator 101 without the need for an extension. Accordingly, the lead 711 can include a lead body 725 that carries the first connection portion 641a (with first connection contacts 633a) and the second connection portion 641b (with second connection contacts 633b). An advantage of this arrangement is that the lead 711 can be implanted in patient without an extension, e.g., in patients for whom the lead 711 and signal generator 101 are located close enough to each other eliminate the need for an extension. A potential drawback is that the needle (or other device) used to insert the lead 711 at its target location will typically need to be configured to accommodate the multiple connection portions 641a, 641b. For example, the needle may be configured to split so as to be removed over the additional width presented by the multiple connection portions 641a, 641b.

One feature of several of the embodiments described above is that the close contact spacing in the central portion of the lead can allow the practitioner to center the lead over a target location while providing a multitude of signal delivery contacts that can be used to pinpoint a target neural population. One particular advantage associated with this arrangement is that it can allow the practitioner to maintain therapeutic efficacy, even if the lead migrates after it is implanted in the patient. For example, the inventor has found that in at least one clinical setting, anecdotally, about 80-100% of patients implanted with a conventional lead initially reported a successful therapeutic outcome during a trial period. By the end of the trial period, after the signal generator was implanted and either reconnected to the lead, or connected to a replacement lead, the number of patients reporting a successful therapeutic outcome may have reduced to about 60%, with the remaining 40% requiring additional intervention (e.g., lead re-positioning and/or contact re-programming) to obtain a positive therapeutic result. With a wider selection of closely spaced contacts the practitioner can more quickly identify and program pairs of contacts in the event the lead migrates during the course of the trial period and/or after a permanent implant. This result can accordingly save both the patient and practitioner time, and can reduce the amount of time the patient suffers from pain that the signals directed from the lead address.

Another advantage of certain embodiments having the foregoing features is that the combination of closely and more widely spaced contacts can approximate a normal distribution. While the significant majority of patients will be treated with the intermediate signal delivery contacts, the distal-most and proximal-most contacts remain available for treating the remaining (e.g., “outlier”) patients. Still another advantage of certain embodiments having the foregoing features is that the arrangement of sixteen signal delivery contacts is compatible with existing signal generators having sixteen channels, and does not result in a lead having so many internal conductors (e.g., 32) that it becomes too stiff to implant successfully in at least some patients.

### 3.0 Additional Embodiments

A spinal cord stimulation system in accordance with an embodiment of the present technology includes an implantable signal generator programmed with instructions that, when executed, deliver a non-paresthesia-producing therapy signal having a frequency in a frequency range from 1.5 kHz to 100 kHz. The system further includes a lead body having a proximal portion and a distal portion, and a plurality of signal delivery contacts carried by the distal portion of the lead body. The signal delivery contacts include multiple distal-most signal delivery contacts spaced apart from each other by a first distance, multiple proximal-most signal delivery contacts spaced apart from each other by a second distance, and multiple intermediate signal delivery contacts spaced apart from each other by a third distance less than the first distance and less than the second distance. A plurality of connection contacts are carried by the proximal portion of the lead body, and a plurality of conductors are carried by the lead body, with individual conductors connected between an individual connection contact and an individual signal delivery contact.

A representative method in accordance with embodiments of the present technology includes programming an implanted signal generator to direct a non-paresthesia-producing therapy signal to a patient's spinal cord region at a vertebral level aligned with the patient's T9-T10 disc. The therapy signal has a frequency in a frequency range of 1.5 kHz to 100 kHz, and the therapy signal is directed to at least two contacts of a lead coupled to the implanted signal generator. The lead has sixteen signal delivery contacts carried by the distal portion of the lead body, with each signal delivery contact having a width of three millimeters, and with three distal-most signal delivery contacts being spaced about from each other by three millimeters, three proximal-most signal contacts spaced apart from each other by three millimeters, and ten intermediate signal delivery contacts spaced apart from each other by one millimeter.

In other embodiments, the spinal cord stimulation system and/or associated methods can include other features. For example, the intermediate signal delivery contacts can be spaced apart by a distance of three millimeters or less, two millimeters or less, or one millimeter. The first and second distances can be the same or different, depending upon the embodiment. The stimulation system can include a lead body in a particular embodiment, and a lead body connected or connectable to an extension in another embodiment.

From the foregoing, it will be appreciated that specific embodiments of the disclosed technology have been described herein for purposes of illustration, but that various modifications may be made without deviating from the technology. For example, in some embodiments, the lead system can have numbers of signal delivery contacts other than those expressly disclosed herein. In still further embodiments, the signal delivery contacts can have spacings and/or widths other than those expressly disclosed herein. The implantable signal generator can include two connection ports in particular embodiments, and other numbers of connection ports in other embodiments. The signal delivery contacts can be electrically coupled to each other during use to form of bipoles, or other contact combinations (e.g., tripoles). Each signal delivery contact can be connected to a corresponding connection or extension contact with an individual conductor in some embodiments, and in other embodiments, multiple signal delivery contacts can be “ganged” together. As discussed above, while the lead system may include proximal, distal, and intermediate contacts, the practitioner may in some cases use only the intermediate contacts. The signal generator can be separable from the lead system, or integrated with the lead system, and can receive energy from an implanted energy source (e.g., a battery or a capacitor) and/or an external energy source (e.g., a transmitter located outside the body) that transmits energy to the signal generator and/or directly to the lead system via an inductive or other transdermal link.

Certain aspects of the technology described in the context of particular embodiments may be combined or eliminated in other embodiments. For example, in at least some embodiments, the proximal and distal contacts can be eliminated. Further, while advantages associated with certain embodiments of the disclosed technology have been described in the context of those embodiments, other embodiments may also exhibit such advantages, and not all embodiments need necessarily exhibit such advantages to fall within the scope of the present technology. Accordingly, the disclosure and associated technology can encompass other embodiments not expressly shown or described herein.

To the extent any materials incorporated herein conflict with the present disclosure, the present disclosure controls.

I/we claim:

1. A spinal cord stimulation system, comprising:
an implantable signal generator having first and second lead connection ports and being programmed with instructions that, when executed, deliver a non-paresthesia-producing therapy signal having a frequency in a frequency range from 1.5 kHz to 100 kHz;
a lead body having a proximal portion and a distal portion;
sixteen signal delivery contacts carried by the distal portion of the lead body, with each signal delivery contact having a contact width of three millimeters, and wherein, of the sixteen signal delivery contacts,
the three distal-most signal delivery contacts are spaced apart from each other by three millimeters,
the three proximal-most signal delivery contacts are spaced apart from each other by three millimeters, and
the ten intermediate signal delivery contacts are spaced apart from each other by one millimeter;
sixteen connection contacts carried by the proximal portion of the lead body;
sixteen conductors carried by the lead body, with individual conductors connected between an individual connection contact and an individual signal delivery contact; and
a lead extension having a distal portion with a connector positioned to connect to the sixteen connection contacts of the lead body, the lead extension further having a proximal portion, the proximal portion being bifurcated to include a first connection portion and a second connection portion, the first connection portion carrying a first set of eight extension contacts positioned to be received in a first connection port of the implantable signal generator, the second connection portion carrying a second set of eight extension contacts positioned to be received in a second connection port of the implantable signal generator.

2. The system of claim 1 wherein the first connection port of the implantable signal generator includes eight terminals and the second connection port of the implantable signal generator includes eight additional terminals.

3. A spinal cord stimulation system, comprising:
an implantable signal generator having first and second lead connection ports and being programmed with instructions that, when executed, deliver a non-paresthesia-producing therapy signal having a frequency in a frequency range from 1.5 kHz to 100 kHz;
a lead body having a proximal portion and a distal portion, the proximal portion being bifurcated to include a first connection portion and a second connection portion;
sixteen signal delivery contacts carried by the distal portion of the lead body, with each signal delivery contact having a contact width of three millimeters, and wherein, of the sixteen signal delivery contacts,
the three distal-most signal delivery contacts are spaced apart from each other by three millimeters;
the three proximal-most signal delivery contacts are spaced apart from each other by three millimeters; and
the ten intermediate signal delivery contacts are spaced apart from each other by one millimeter;
sixteen connection contacts carried by the proximal portion of the lead body, with:
a first set of eight connection contacts carried by the first connection portion;
a second set of eight connection contacts carried by the second connection portion; and
sixteen conductors carried by the lead body, with individual conductors connected between an individual connection contact and an individual signal delivery contact.

4. The system of claim 3 wherein a first connection port of the implantable signal generator includes eight terminals, and a second connection port of the implantable signal generator includes eight additional terminals.

5. A spinal cord stimulator, comprising:
a lead system having a proximal portion and a distal portion;
a plurality of signal delivery contacts carried by the distal portion of the lead system, with:
multiple distal-most signal delivery contacts being spaced apart from each other by first distance;
multiple proximal-most signal delivery contacts being spaced apart from each other by a second distance; and
multiple intermediate signal delivery contacts being spaced apart from each other by a third distance less than the first distance and less than the second distance;
a plurality of connection contacts carried by the proximal portion of the lead system; and
a plurality of conductors carried by the lead system, with individual conductors connected between an individual connection contact and an individual signal delivery contact.

6. The stimulator of claim 5 wherein the lead system includes three distal-most signal delivery contacts, three proximal-most signal delivery contacts, and ten intermediate signal delivery contacts.

7. The stimulator of claim 5 wherein each signal delivery contact has a width of three millimeters.

8. The stimulator of claim 5 wherein neighboring intermediate signal delivery contacts are spaced apart by three millimeters or less.

9. The stimulator of claim 5 wherein neighboring intermediate signal delivery contacts are spaced apart by two millimeters or less.

10. The stimulator of claim 5 wherein neighboring intermediate signal delivery contacts are spaced apart by one millimeter.

11. The stimulator of claim 5 wherein the first and second distances are equal.

12. The stimulation system of claim 5 wherein the lead system includes a lead body.

13. The stimulator of claim 5 wherein the lead system includes a lead body, and further includes:
a lead extension having a distal portion with a connector positioned to receive the plurality of connection contacts, and a proximal portion, the proximal portion being bifurcated to include a first connection portion and a second connection portion, the first connection portion carrying a first set of extension contacts positioned to be received in a first connection port of an implantable signal generator, the second connection portion carrying a second set of extension contacts positioned to be received in a second connection port of the implantable signal generator.

14. The stimulator of claim 5 wherein the lead system includes a lead body having the proximal portion and the distal portion, and wherein the proximal portion is bifurcated to include a first connection portion and a second connection portion, the first connection portion carrying a first set of connection contacts positioned to be received in a first connection port of an implantable signal generator, the second connection portion carrying a second set of connection contacts positioned to be received in a second connection port of the implantable signal generator.

15. A method for configuring a patient treatment system, comprising:
programming an implantable signal generator to direct a non-paresthesia-producing therapy signal to a patient's spinal cord region at a vertebral level aligned with the patient's T9-T10 disc, wherein
the therapy signal has a frequency in a frequency range from 1.5 kHz to 100 kHz, and
the therapy signal is directed to at least two contacts of a lead system coupled to the implanted signal generator, the lead system having sixteen signal delivery contacts carried by the distal portion of the lead body, with each signal delivery contact having a width of three millimeters, and wherein, of the sixteen signal delivery contacts,
the three distal-most signal delivery contacts being spaced apart from each other by three millimeters;
the three proximal-most signal delivery contacts being spaced apart from each other by three millimeters; and
the ten intermediate signal delivery contacts being spaced apart from each other by one millimeter.

16. The method of claim 15 wherein the at least two contacts includes a bipole pair of contacts.

17. The method of claim 15 wherein programming includes programming the implantable signal generator to direct the therapy signal to only intermediate signal delivery contacts.

18. The method of clam 15, further comprising positioning the lead system with at least some of the intermediate contacts aligned along a rostral-caudal axis with the T9-T10 disc.

19. The method of claim 15 wherein the lead system include a lead body, and wherein the method further comprises connecting the lead body directly to the implantable signal generator.

20. The method of claim 15 wherein the lead system include a lead body, and wherein the method further comprises connecting the lead body to an extension, and connecting the extension to the implantable signal generator.

21. A method for treating a patient, comprising:
programming an implantable signal generator to direct a non-paresthesia-producing therapy signal to a patient's spinal cord region, wherein:
the therapy signal has a frequency in a frequency range from 1.5 kHz to 100 kHz, and
the therapy signal is directed to at least two contacts of a lead system coupled to the implanted signal generator, the lead system having multiple signal delivery contacts carried by the distal portion of the lead system, wherein
multiple distal-most signal delivery contacts being spaced apart from each other by first distance,
multiple proximal-most signal delivery contacts being spaced apart from each other by a second distance, and
multiple intermediate signal delivery contacts being spaced apart from each other by a third distance less than the first distance and less than the second distance.

22. The method of claim 21 wherein the at least two contacts includes a bipole pair of contacts.

23. The method of claim 21 wherein programming includes programming the implantable signal generator to direct the therapy signal to only intermediate signal delivery contacts.

24. The method of claim 21 wherein the intermediate signal delivery contacts are spaced apart from each other by less than three millimeters.

25. The method of claim 21 wherein the intermediate signal delivery contacts are spaced apart from each other by less than two millimeters.

26. The method of claim 21 wherein the intermediate signal delivery contacts are spaced apart from each other by one millimeter.
